(19) United States
(12) Patent Application Publication
(10) Pub. No.: US 2023/0172822 A1
(43) Pub. Date: Jun. 8, 2023

(54) FRAGRANT COMPOSITION

(71) Applicant: LVMH RECHERCHE, SAINT JEAN DE BRAYE (FR)

(72) Inventors: Elodie CHAMPREDONDE, SAINT JEAN DE BRAYE (FR); Valérie BOUCHARD DE LA POTERIE, SAINT JEAN DE BRAYE (FR)

(21) Appl. No.: 17/997,459

(22) PCT Filed: Apr. 30, 2021
(86) PCT No.: PCT/FR2021/050749

§ 371 Date: Oct. 28, 2022

(30) Foreign Application Priority Data
Apr. 30, 2020 (FR) 2004314

(51) Int. Cl. A61K 8/31; A61Q 13/00; A61K 8/06

(52) U.S. Cl. CPC A61K 8/31; A61Q 13/00; A61K 8/062; A61K 2800/30; A61K 2800/87

(57) ABSTRACT

The invention relates to a liquid perfuming cosmetic composition comprising a) at least 50% by mass of water, b) less than 10% by mass of ethanol, c) at least 3% by mass of a perfume concentrate and d) at least one oil chosen from alkanes, and d) less than 0.1% by mass of an emulsifying agent, the percentages being expressed relative to the mass of the composition.

### TECHNICAL FIELD

The present invention relates to a new perfuming cosmetic composition which may be an eau de toilette, an eau de cologne, a perfume extract, an esprit de parfum or an eau de parfum depending on the content of perfume concentrate it contains and the notes it expresses. This perfuming composition has excellent properties such as the olfactory fidelity of the perfume concentrate and the persistence of the scented note over time after application, the absence of a sticky to touch feeling, and the ability to be sprayed.

In order to manufacture perfuming compositions that contain water, dispersing the perfume concentrate in water is not possible because the concentrate hydrolyzes and develops a prohibitive odor: the ester functions of the molecules present in the concentrate hydrolyze in the presence of water to form acids which develop an acetic odor characteristic of these functions. This is why, in order to prepare hydro-alcoholic perfuming compositions, a perfume concentrate is dissolved in an alcohol such as ethanol, before being dispersed in water.

However, the use of alcohol is questioned because of risks of intolerance, allergy, irritation, photosensitization in some users, and safety risks associated with the use of alcohol. In addition, certain religious customs prohibit its use.

A first way of preparing an alcohol-free perfuming composition is to use other compounds in order to disperse the perfume concentrate in water, for example a solubilizing agent and an emulsifying agent. However, solubilizers and emulsifiers have a negative influence on the olfactory and organoleptic properties of the product: the olfactory note may be altered over time and the feel of the composition may give a sticky sensation. In order to avoid these drawbacks, solubilizers and emulsifiers of perfuming concentrate must be used in small quantities, which limits the rate of perfume concentrate that can be introduced into a perfuming composition. This is the case of the alcohol-free compositions described in application FR 2 952 531 which contain a solubilizing agent, an emulsifying agent and small amounts of perfume concentrate, typically around 2%.

A second way to eliminate alcohol in a perfuming composition while preserving the stability of the concentrate is to eliminate water. Perfuming compositions without anhydrous alcohol are oily compositions, sticks, powders or essences. These products have viscous solid or liquid textures, which are very different from conventional alcoholic or hydro-alcoholic compositions. These compositions are not sprayable, can stain clothing, and can only be used by direct application to the skin, which constitutes a real limitation of use. They therefore require adapting the packaging and the application gesture, which is not always accepted by the consumer. In addition, such compositions are perceived as oily and do not provide any freshness.

In view of all the disadvantages listed above, the need remains to provide an aqueous perfuming composition containing high contents of perfume concentrate and reduced, or even zero, contents of ethanol, and which has organoleptic properties greater than or equal to those of the compositions of the prior art.

The need remains in particular to provide a perfuming composition containing high levels of perfume concentrate which has good diffusion intensity and/or good persistence, which is sprayable, whose olfactory identity is stable over time without degradation of the perfuming concentrate, and/or whose feel is non-greasy and non-sticky. The invention aims to achieve at least one of the aforementioned objectives.

General Description of the Invention

The invention therefore provides a liquid perfuming cosmetic composition comprising a) at least 50% by mass of water, b) less than 10% by mass of ethanol, c) at least 3% by mass of a perfume concentrate and d) at least one oil chosen from alkanes, and d) less than 0.1% by mass of an emulsifying agent, the percentages being expressed relative to the mass of the composition.

### BRIEF DESCRIPTION OF THE DRAWINGS

FIG. 1 shows the evolution of the olfactory intensity (MD BB) of a composition of the invention and of a control over time at TO, T1 and T2.

FIG. 2 shows the evolution of the olfactory fidelity (MD BB) of a composition of the invention and of a control over time at TO, T1 and T2.

### DESCRIPTION OF EMBODIMENTS

A “perfuming composition” or “perfume” is defined as a product in liquid form intended to perfume an individual after it has been sprayed or applied to the skin, hair or clothing. Such a product is not rinsed off after application. A perfuming composition will thus be distinguished from a perfumed composition. A cosmetic composition can be perfumed without being perfuming.

In the context of the invention, the terms “from . . . to” are intended to include the value range limits, unlike the terms “between . . . and” which are intended to exclude the value range limits. Preferably, the composition according to the invention has two visually distinct phases on standing.

It can be in the form of a dual phase comprising two phases visually distinct that are immiscible on standing (preferably an aqueous phase and an oily phase), which mix easily by agitation to allow an homogeneous application, and which quickly phase out after stopping agitation to return to their initial state. Preferably, the composition has two superimposed phases visually distinct, having preferably a sharp interface. More preferably, one of the phases, preferably both phases are transparent.

The composition contains a perfume concentrate, also called “perfuming concentrate”. The perfume concentrate may for example be chosen from compounds whose INCI name appears on the list of ingredients of a perfuming composition available for sale under the name “perfume”. A perfume concentrate is a compound or a mixture of compounds at least partially volatile at room temperature, the smell of which is detected. A perfume concentrate made up of essential oils must necessarily be diluted to express its full olfactory potential, namely a perception changing during the day after application to the skin, dictated by the presence of odorous organic compounds with very different volatilities from each other. The development of a perfume concentrate consists of combining several perfuming materials in order to give to the perfuming composition a top note, a heart note and a base note. A perfume concentrate used in the context of the invention preferably comprises mainly top notes and heart notes.

The perfume concentrate is prepared from natural or synthetic organic perfuming materials.

Perfuming materials of natural origin include, for example, extracts of flowers, stems and leaves, fruits, fruit peels, roots, wood, herbs and grasses, resins and balms. These plant-based perfuming materials can be essential oils, such as, for example, essences of bergamot, rose, lavender, sandalwood, cardamom, sage, chamomile, clove, lemon balm, mint, cinnamon leaves, juniper, vetiver, olibanum, galbanum and labdanum.

Perfuming materials of synthetic origin include, for example, hedione, ethylene brassilate and habanolide, benzyl acetate, benzyl benzoate, phenoxyethyl isobutyrate, p-tert-butylcyclohexyl acetate, citronellyl acetate, citronellyl formate, geranyl acetate, linalyl acetate, dimethyl-benzylcarbinyl acetate, phenylethyl acetate, linalyl benzoate, benzyl formate, ethylmethylphenyl glycinate, alkylcyclohexyl propionate, styralyl propionate and benzyl salicylate, benzylethyl ether, straight alkanals with 8 to 18 carbon atoms, citral, citronellal, citronellyloxyacetaldehyde, cyclamenaldehyde, hydroxycitronellal, ionones such as alpha-isomethylionone, and methylcedrylketone, anethole, citronellol, eugenol, isoeugenol, geraniol, linalool, phenylethylalcohol, terpineol, terpenes. These compounds are often in the form of a mixture of two or more of these odorous substances.

Olfactory notes are often classified in families that allow a consumer to know what perception to expect. Mention may be made, for example, of citrus perfumes, aromatics, floral perfumes, musky perfumes, fruity perfumes, spicy perfumes, oriental perfumes, marine perfumes, aquatic notes, chypre perfumes, woody perfumes, ferns, leathers and mixtures thereof.

The perfume concentrate represents at least 3% by mass, preferably at least 4% by mass, for example at least 5% by mass, more preferably at least 6%, 7% or 8% by mass, better at least 10%, 11%, 12%, 13% or 14% by mass, and even better at least 15% by mass of the mass of the composition.

The concentration of the perfume concentrate can range from 3% by mass to 40% by mass or be between 3% and 40% by mass. It preferably ranges from 4% to 38% by mass or from 5% by mass to 35% by mass, for example from 7% by mass to 30% by mass or from 10% by mass to 25% by mass of the mass of the composition. In one embodiment, the perfume concentrate represents from 5% by mass to 15% by mass of the mass of the composition.

The ethanol content of the composition is less than 10% by mass relative to the mass of the composition, preferably less than 5%, and even more preferably less than 2%. The composition is preferably free of ethanol, added during the preparation of the perfuming composition by mixing the various ingredients. It cannot be excluded that there are traces of ethanol in some of the ingredients used.

The composition of the invention contains less than 0.5%, preferably less than 0.1% by mass of an emulsifying agent such as a surfactant or an emulsifying polymer, which could degrade the olfactory and organoleptic properties of the composition. It preferably contains less than 0.05% by mass of an emulsifying agent, and even more preferably less than 0.01% by mass of the composition. According to one particularly preferred mode, the composition is free of it.

In particular, the composition comprises less than 0.5% by mass, preferably less than 0.1% by mass of one or more nonionic surfactants, polyoxyalkylene surfactants comprising at least five units chosen from—CH2CH(OH)CH2- and —OCH2CH2-, such as polyoxyethylene compounds and polyoxypropylene compounds. Among these surfactants, mention may be made of polyoxyalkylene ethers, such as cetyl ether POE(10), polyoxyalkylene esters, such as hydrogenated castor oil PEG-40 or sorbitan monolaurate POE(20), condensates of polyoxyethylene alkylphenols, condensates of ethylene oxide with the reaction product of propylene oxide and ethylene diamine, polyethoxylated alcohols, polysorbates, and dimethicone copolyols. The composition is preferably free of nonionic surfactant.

An emulsifying polymer is defined as an amphiphilic polymer, that is to say endowed with at least one hydrophilic part and at least one hydrophobic part. Hydrophilic groups and hydrophobic groups are well known to those skilled in the art, that could help to form and stabilize emulsions. As examples of emulsifying amphiphilic polymers, mention may be made of polymers and copolymers based on AMPS, crosslinked or non-crosslinked, polyacrylic acid/alkyl acrylate copolymers, in particular acrylate/C10-C30-alkylacrylate copolymers and mixtures thereof. The composition preferably comprises less than 0.5% by mass, preferably less than 0.1% by mass of one or more emulsifying polymers.

The composition is preferably devoid of any emulsifying agent.

The oil is chosen from alkanes, preferably linear or branched C8-C19 alkanes, hydrogenated polyisobutenes, squalane, hemisqualane and mixtures thereof. An alkane is defined as a compound made up of carbon and hydrogen.

Preferably, the alkane oil is volatile. A “volatile oil” is defined as an oil having lost more than 20% by mass of its mass after 15 minutes, more than 40% by mass of its mass after 30 minutes and more than 70% by mass of its mass after 60 minutes, according to the following protocol:

20 mg of the oil to be studied is weighed on a 5 cm×5 cm PMMA plate using a micropipette and a precision scale. This material is spread with the finger over the entire plate. Then, it is placed in a ventilated enclosure thermostated at 25° C. and 50% humidity. For each material, the test is carried out 3 times. The mass loss during drying is measured after 15 minutes, 30 minutes and 60 minutes. The lost mass is expressed according to the following calculation:

% lost mass=mₜ₀−mₜₓ/mₜ₀×100 [Math.1]
wherein mtx corresponds to the mass remaining at the measured time (t15 min, t30 min or t60 min) and mt0 corresponds to the mass initially applied.

Oil volatility measurements are expressed in time (in minutes). The person skilled in the art will thus be able to define the oils which are suitable for the invention on the basis of this test for monitoring its mass loss with respect to time.

As alkane oil, mention may be made in particular of:
branched C8-C16 alkanes such as C8-C16 iso-alkanes (also called isoparaffins), isododecane, isodecane, isohexadecane, and for example the oils sold under the trade names Isopars or Permetyls,
linear or branched alkanes having hydrocarbon chains in
C9-C17, C10-C14, such as a mixture of undecane and tridecane, marketed by BASF Care Creations under the name Cetiol® Ultimate,
C15-C19, such as those marketed by Seppic under the name Emogreen® L15,
C9-C12, C12-C14, such as those marketed by BIOSYNTHIS under the name Vegelight® Silk (INCI name C9-12 ALKANE), Vegelight® 1214LC,
n-dodecane (C12) and ntetradecane (C14), in particular sold by Sasol respectively under the references PARAFOL® 12-97 and PARAFOL® 14-97, and mixtures thereof.

Mention may also be made of squalane, hemisqualane of plant, mineral or synthetic origin such as, for example, plant-derived hemisqualane such as the one marketed by the company Amyris under the reference Neossance® hemisqualane, plant-derived squalane marketed for example under the name Squalive® by the company Biosynthis, or even hydrocarbons such as hydrogenated polyisobutene having a molecular weight of less than 250 g/mol, such as the one bearing the INCI name HYDROGENATED POLYISOBUTENE, such as the products with the trade names Parleam®, in particular PARLEAM®3 or the product Polysynlane®4 sold by the company NOF CORPORATION, and mixtures thereof.

The alkane oil is advantageously present in an amount sufficient to solubilize the perfume concentrate.

The alkane oil preferably represents at most 20% by mass, preferably at most 15% by mass, more preferably at most 10% by mass, and particularly preferably at most 5% by mass of the mass of the composition. It can range from 1% to 10% by mass or be between 1% and 10% by mass. It preferably ranges from 1% to 8% by mass or from 2% to 7% by mass, and even better from 2% to 5% by mass of the mass of the composition. In one embodiment, the composition contains from 1% to 5% by mass of a volatile alkane oil or of a mixture of volatile alkane oils, when the composition contains several of them.

The mass ratio between the alkane oil and the perfume concentrate is advantageously greater than a minimum value chosen from the group consisting of 1:20, 1:10, 1:5, and less than a maximum value chosen from the group consisting of 3:1, 2:1, 1:1 and 1:2. In one embodiment, the mass ratio between the alkane oil and the perfume concentrate ranges from 1:10 to 2:1 or from 1:5 to 1:1.

The composition according to the invention comprises at least 50% by mass of water, preferably at least 60% by mass, and better at least 65% by mass. The water content of the composition may thus be greater than a minimum value equal to 70%, 75%, 80% or 85% by mass relative to the mass of the composition. The water content ranges for example from 75% to 95% by mass of the mass of the composition.

It is preferred that the sum of the mass percentages of the water, the perfume concentrate and the alkane oil be greater than or equal to 95% by mass, more preferably still greater than 98% by mass of the mass of the composition.

Advantageously, the composition of the invention essentially consists of water, perfume concentrate and alkane oil. By “essentially consists of”, it is meant that the sum of the percentages of water, perfume concentrate and alkane oil is greater than or equal to 98% by mass, or even greater than or equal to 99% by mass.

In addition to the ingredients described above, the composition may contain at least one cosmetically acceptable ingredient chosen from colorants, preservatives, pH adjusters, electrolytes, UV filters, and antioxidants.

These additional ingredients must be olfactory neutral so as not to alter the smell of the perfuming composition, nor cause a color change, the appearance of a disturbance associated with stability problems.

The cosmetic active agents can, for example, be advantageously chosen from those having a skincare action, such as anti-ageing active agents, moisturizing agents or any other cosmetically active agent whose addition to the composition of the invention makes it possible to obtain a beneficial effect for the skin while preserving the organoleptic properties of the composition.

Colorants are for example: caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, Ferric Ferrocyanide, Ultramarines, Acid Yellow 3/Yellow 10, Acid Blue 3, Yellow 10. Fat-soluble dyes are e.g. Sudan Red, D&C Red 17, D&C Green 6, Beta-carotene, soybean oil, Sudan brown, D&C Yellow 11, D&C Violet 2, D&C Orange 5, quinoline yellow, annatto. The colorants generally represent from 0.01 to 1%, preferably from 0.05 to 0.5%, by mass of the mass of the perfuming composition.

Among antioxidants and preservatives, mention may be made, for example, of ascorbic acid, di-tert-butyl-p-hydroxytoluene (also called BHT or 2,6-di-tert-butyl-p-cresol), BHA (tert-butyl-4-hydroxyanisole), tocopherols such as vitamin E, tocopherol derivatives such as tocopheryl acetate, gallic acid and derivatives thereof, potassium sorbate, sodium benzoate, hydroxyacetophenone, and chlorophenesin.

According to a particular embodiment, the perfuming composition contains from 8% to 12% of perfume concentrate, from 1% to 5% of alkane oil(s), and water in an amount such that the sum of the concentrations of perfume concentrate, alkane oil(s) and water is greater than 99%, the percentages being expressed by mass relative to the mass of the composition.

The composition may optionally contain, in addition to the ingredients described above, an additional oily compound, a thickening agent or a powder. In one embodiment of the invention, the composition of the invention does not include any of these ingredients.

The composition contains an aqueous phase and a fatty phase, the fatty phase containing an alkane oil and a perfume concentrate. It is preferred that the fatty phase contain less than 1% by mass, preferably less than 0.1% by mass, of an additional oily compound.

The perfuming composition may be packaged in a container optionally fitted with an application means. The application means can be a spraying means, a ball or a cap.

The invention also relates to a vial provided with an application means and a packaging means, and containing the composition described above. The packaging means is preferably transparent in order to reveal the composition of the invention, which is also preferably transparent. The application means can be a manual pump, a ball or a cap. The compositions can be applied in the form of fine droplets using pressurization devices. These devices are well known to those skilled in the art and include non-aerosol pumps or “atomizers”, aerosol containers comprising a propellant as well as aerosol pumps using compressed air as propellant.

The compositions and vials of the invention may be in the form of eau de toilette, eau de cologne, perfume extract, voile de parfum, esprit de parfum or eau de parfum. In accordance with the knowledge of those skilled in the art, an eau de cologne generally contains 3% to 8%, an eau de toilette generally contains 5% to 15%, an eau de parfum generally contains 12% to 20%, a perfume extract or an esprit de parfum generally contains more than 15%, and a voile de parfum generally contains from 5% to 8%, of perfume concentrate, the percentages being expressed by mass relative to the mass of the composition.

Another object of the invention is a cosmetic process comprising a step of applying a cosmetic composition described above to the keratin materials or clothing of an individual. This process can be a process for perfuming the skin or the hair of an individual, which consists in applying, using a spraying means, to the skin or the hair of the individual, the composition described above. The composition can also be applied to clothing; however, it is preferred to apply it directly to the skin, preferably on a part of the body that is not the face. The invention will be illustrated in more detail by the following examples.

### Example 1: Evaluation of Olfactory Fidelity and Persistence of a Perfuming Composition According to the Invention

The following perfuming composition was prepared according to the invention.

TABLE 1

| | Ingredients | % by mass |
|---|---|---|
| Aqueous | Water | 86.7 |
| phase | Hydroxyacetophenone | 0.5 |
| | Chlorphenesin | 0.3 |
| Oily | C9-12 alkanes | 2.5 |
| phase | (Cetiol Ultimate ® from BASF) | |
| | Perfume concentrate | 10 |

The raw materials of the aqueous phase and the fatty phase were weighed and mixed, respectively, before pouring the fatty phase into the aqueous phase.

The perfuming composition is packaged in a vial fitted with a spray device. The composition appears on standing in the form of a dual phase, of which the two immiscible transparent phases are visually distinct.

After manual stirring to homogenize the composition of the composition, the latter is packaged in a vial comprising a spray device.

The olfactory fidelity and the intensity (persistence) of the composition according to the invention are evaluated by a panel of four experts with experience in objective and descriptive characterizations of the sensory qualities of perfumed or perfuming cosmetic compositions.

A commercial perfume of the “floral” type and of medium persistence, which contains 79% ethanol (MISS DIOR BLOOMING BOUQUET®), and a floral perfume concentrate, is used as a Reference. The example of the invention is prepared with the same concentrate as that of the Reference.

For each product, the intensity and fidelity are assessed immediately after application to the skin (TO), then after 6 hours (T1) and 8 hours (T2). For each criterion, the expert gives a score on a scale of 0 to 9 (optimal score).

The protocol is as follows:
a single spray of the composition of the invention is applied, approximately 5 cm from the arm, to the skin of one forearm of the expert, and the same application is carried out with the Reference on the other forearm,
after evaluation at TO, the two scented skin areas are protected from friction
at T1 then at T2, these same criteria are evaluated again.

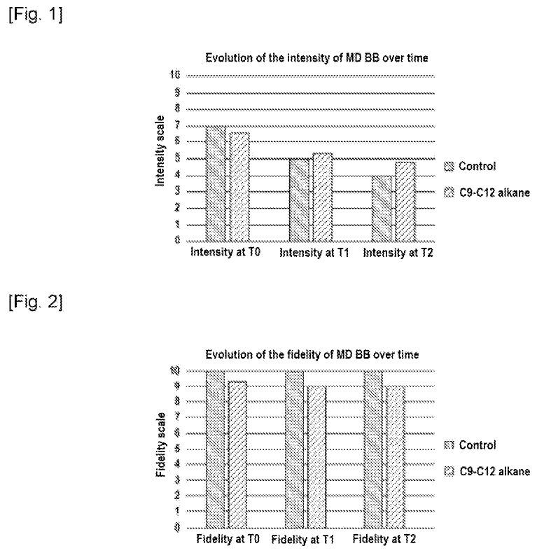

The results have been reported in FIGS. 1 and 2.

The test with C9-C12 alkanes is more persistent than the control after 6 and 8 hours. It is less fusing and less fresh than the control at TO. The accuracy of the test with C9-C12 alkanes to the control is constant over time.

### Example 2

The following perfuming composition was prepared according to the invention.

TABLE 2

| | Ingredients | % by mass |
|---|---|---|
| Aqueous phase | Water | 87.1 |
| | Citric acid | 0.1 |
| | Potassium sorbate | 0.2 |
| | Sodium benzoate | 0.1 |
| Oily phase | Hydrogenated polyisobutene | 2.5 |
| | (POLYSYNLANE ® 4) | |
| | Perfume concentrate | 10 |

The perfume concentrate used in this example is the same as the one used to prepare the commercial product MISS DIOR BLOOMING BOUQUET®.

The raw materials of the aqueous phase and the fatty phase respectively were weighed and mixed, before pouring the fatty phase into the aqueous phase.

The perfuming composition is packaged in a vial fitted with a spray device. The composition appears on standing in the form of a dual phase, of which the two immiscible transparent phases are visually distinct.

After manual stirring to homogenize the composition of the composition, the latter is packaged in a vial comprising a spray device.

The composition is thus sprayed onto the skin or clothing without the olfactory identity being altered by the presence of an alkane oil.

### Examples 3 and 4

The following four compositions were prepared: two formulas in accordance with the prior art described in application FR 2 952 531, and two compositions according to the invention.

TABLE 3

| | Comparative | Comparative | Example | Example |
|---|---|---|---|---|
| | formula 1 | formula 2 | 3 | 4 |
| Cross-linked | 0.8 | 0.8 | 0 | 0 |
| copolymer of | | | | |
| AMPS and ester | | | | |
| of (meth)acrylic | | | | |
| acid and fatty | | | | |
| alcohol | | | | |
| (Aristoflex ® | | | | |
| HMS) | | | | |
| Perfume | 2 | 2 | 4 | 8 |
| Preservative | 0.2 | 0.2 | 0.2 | 0.2 |
| N-dodecane | 10 | | | |
| C9-12 ALKANE | | 10 | 10 | 10 |
| Water | Qsp 100 | Qsp 100 | Qsp 100 | Qsp 100 |

Comparative formulas 1 and 2 have a thick texture and are opaque white, while the compositions of examples 3 and 4 are sprayable and transparent.

1-14. (canceled)

15. A liquid perfuming cosmetic composition comprising a) at least 50% by mass of water, b) less than 10% by mass of ethanol, c) at least 3% by mass of a perfume concentrate and d) at least one oil chosen from alkanes, and d) less than 0.1% by mass of an emulsifying agent, the percentages being expressed relative to the mass of the composition.

16. The composition according to claim 15, wherein the alkane oil represents from 1% to 10% by mass of the composition.

17. The composition according to claim 15, which comprises from 8% by mass to 12% by mass of perfume concentrate, from 1% by mass to 5% by mass of alkane oil, and water in an amount such that the sum of the concentrations of perfume concentrate, oil and water is greater than 99% by mass, the percentages being expressed relative to the mass of the composition.

18. The composition according to claim 15, wherein the oil is chosen from C8-C19 alkanes, hydrogenated polyisobutenes, squalane, hemisqualane and mixtures thereof.

19. The composition according to claim 15, wherein the mass ratio between the alkane oil and the perfume concentrate ranges from 1:10 to 2:1.

20. The composition according to claim 15, wherein the oil is chosen from: branched C8-C16 alkanes,
linear or branched alkanes having hydrocarbon chains in C9-C17, C10-C14, C15-C19, C9-C12, C12-C14, n-dodecane (C12), n-tetradecane (C14),
squalane, hemisqualane,
hydrocarbons having a molecular weight of less than 250 g/mol, and
mixtures thereof.

21. The composition of claim 20, wherein the branched C8-C16 alkanes are selected from C8-C16 iso-alkanes.

22. The composition of claim 21, wherein the branched C8-C16 alkanes are selected from the group consisting of isododecane, isodecane, and isohexadecane.

23. The composition of claim 20, wherein the hydrocarbon hydrogenated polyisobutene having a molecular weight of less than 250 g/mol is hydrogenated polyisbutene.

24. The composition according to claim 15, wherein the alkane oil is volatile.

25. The composition according to claim 15, wherein it comprises less than 0.05% by mass of a surfactant of the mass of the composition.

26. The composition according to claim 15, wherein it comprises at least 60% by mass of water relative to the mass of the composition.

27. The composition according to claim 15, wherein the ethanol content is less than or equal to 5% by mass.

28. The composition according to claim 15, wherein it is free of ethanol.

29. The composition according to claim 15, wherein it has two visually distinct phases on standing.

30. Vial provided with an applicator and a package, said package containing the perfuming composition according to claim 15.

31. Cosmetic process comprising a step of applying to the keratin materials or clothing of an individual a cosmetic composition according to claim 15.
